(19) United States
(12) Patent Application Publication
(10) Pub. No.: US 2008/0306441 A1
(43) Pub. Date: Dec. 11, 2008

(54) NON-BUCKLING BALLOON CATHETER WITH SPRING LOADED FLOATING FLEXIBLE TIP

(75) Inventors: Hilbert D. Brown, Winston-Salem, NC (US); Steven K. Chen, Westfield, IN (US); Kenneth C. Kennedy, II, Clemmons, NC (US)

Correspondence Address: BRINKS HOFER GILSON & LIONE/CHICAGO/COOK, PO BOX 10395, CHICAGO, IL, 60610, US

(73) Assignee: Wilson-Cook Medical Inc., Winston-Salem, NC (US)

(21) Appl. No.: 12/098,859

(22) Filed: Apr. 7, 2008

Related U.S. Application Data
(60) Provisional application No. 60/922,769, filed on Apr. 10, 2007.

(51) Int. Cl. A61M 25/10; A61M 25/18

(52) U.S. Cl. 604/99.01; 604/103.09; 604/103

(57) ABSTRACT

A balloon catheter including an inflatable balloon affixed to a catheter. The proximal end of the balloon is affixed to the distal end of the catheter so as to provide an air tight seal there between. A stiffening member extends distally of the distal end of the catheter and forms a slip joint connection with the distal end of the balloon to permit the distal end of the balloon to axially move or translate relative to the distal end of the catheter. The slip joint allows the axial length of balloon to change during inflation or deflation without transferring tensile or compressive forces between the balloon and the catheter, thereby preventing transverse creases from forming in the surface of the balloon and preventing the catheter from bowing. The stiffening member provides alignment and lateral support to the distal end of the balloon.

### CROSS-REFERENCE TO RELATED APPLICATIONS

This application claims the benefit of U.S. Provisional Application Ser. No. 60/922,769, filed Apr. 10, 2007, entitled “Non-Buckling Balloon Catheter With Spring Loaded Flexible Tip”, the entire contents of which are incorporated by reference.

### TECHNICAL FIELD

This invention relates to medical devices, and more particularly to balloon catheters that can be placed within a body lumen and inflated to perform various medical procedures. The invention is especially relevant to balloon catheters with balloons formed of non-elastomeric films or materials, wherein the film that forms the balloon is folded and unfolded during deflation and inflation, respectively, of the balloon.

### BACKGROUND OF THE INVENTION

Balloon catheters are used to perform various medical procedures wherein the balloon is positioned within a body lumen or canal and subsequently inflated. In some of these medical procedures, such as in an angioplasty procedure, the balloon is inflated so as to expand the interior volume of the body canal. In this type of procedure, the balloon is expanded to apply pressure to the interior surface of the body canal to thereby compress any tissue protruding into the canal and thereby enlarge the interior volume thereof. Once the tissue has been compressed, and the body canal widened, the balloon is deflated and removed.

In other types of medical procedures, such as photodynamic therapy (PDT), a balloon catheter is used to align and stabilize the catheter within the body lumen. For example, the balloon catheter may be inflated under low pressure within a body lumen such as the esophagus. A therapeutic fiber optic device is then inserted into the catheter in the vicinity of the balloon. The therapeutic fiber optic device is then used to emit light waves to treat the surrounding tissue. In this procedure, the balloon is used to both align the catheter in the center of the body lumen, and to prevent the catheter from moving during the PDT procedure. However, the tissue to be treated must not be unduly compressed by the expanded balloon. Thus, the balloon is expanded only enough to lightly contact the interior surface of the lumen and align the catheter.

As will be explained below, conventional balloon catheters have a number of shortcomings that make them inadequate for many of the above-described procedures, and in particular, for PDT procedures.

A typical balloon catheter 10 is shown in FIGS. 1A-1D. As best seen in FIG. 1A, a conventional balloon catheter 10 comprises a balloon 12 that is affixed to a catheter 14. The balloon 12 is typically manufactured from a non-elastomeric material (e.g., a semi-rigid or non-compliant material), and includes a distal neck or end 16, a proximal neck or end 18 and a central portion 20. The balloon 12 is affixed to the catheter 14 by inserting the distal end 22 of the catheter 14 into and through the proximal end 18 of the balloon 12. The balloon 12 is then slid over the catheter 14 until the distal end 22 of the catheter 14 is inserted into the distal end 16 of the balloon 12. The distal end 22 of the catheter 14 is then affixed to the distal end 16 of the balloon 12 by an adhesive, ultrasonic welding, or some other method. The proximal end 18 of the balloon 12 is similarly affixed to the outer wall of the catheter 14 so as to anchor and seal the proximal end of the balloon 12.

The catheter 14 includes an aperture 24 for the introduction of air or some other fluid into the interior volume of the balloon 12. Although not shown in the drawings, the proximal end of the catheter 14 is typically attached to a devices such as a syringe, that is manipulated to either inflate or deflate the balloon 12 by injecting a fluid into or withdrawing a fluid from, respectively, the interior volume of the balloon 12.

The conventional balloon catheter 10 has a number of drawbacks for use in many of the above-described procedures, and in particular, for use in PDT procedures. When initially manufactured, the balloon catheter 10 generally assumes a shape and configuration as depicted in FIG. 1A. As can be seen in this drawing, the central portion 20 of the balloon 12 is connected to the distal end 16 and the proximal end 18 by tapered or conical sections 26. The tapered sections 26 provide a transition between the larger diameter of the central portion 20 of the balloon 12 and the smaller end portions of the balloon 12 (i.e., the distal end 16 and the proximal end 18) that are connected to the catheter 14.

At the time of packaging by the manufacturer or at the initiation of the medical procedure, the balloon 12 is typically deflated prior to inserting of the balloon catheter 10 into the body canal. Deflation of the balloon 12 is necessary to reduce the overall cross-section or diameter of the device to permit it to pass through an endoscope and/or to navigate and pass through the body's internal canals. FIG. 1B depicts the balloon catheter 10 in the deflated state. As can be seen in this drawing, the balloon 12 is forced to compress in length. This is because the overall length of the material that forms the central portion 20 and the tapered portions 26, as measured along the surface of the balloon 12 in a generally axial direction of the catheter 14 (i.e., from one end of the balloon 12 to the other), is greater than the distance between the distal end 16 and the proximal end 18. As a result of this compression, transverse creases 28 typically form along the surface of the balloon 12.

After the balloon catheter 10 is positioned within the body canal (not shown) at the desired location, inflation of the balloon 12 is initiated as shown in FIG. 1C. As depicted in this drawing, the creases 28 in the surface of the material may prevent the balloon 12 from fully expanding to its normal length (i.e., as shown in FIG. 1A). In other words, the balloon 12 tends to act like a spring under tension. As a result, the portion of the catheter 14 that lies between the distal end 16 and the proximal end 18 of the balloon 12 will be forced into compression, and may begin to bow 30 as a result of these compressive forces.

As inflation of the balloon 12 continues, bowing 30 of the catheter 14 may be increased as shown in FIG. 1D. This is the result of transverse or outward expansion of the central portion 20 of the balloon, which tends to pull the distal end 16 and the proximal end 18 towards each other.

Bowing 30 of the catheter 14 may not be eliminated unless and until a sufficiently high inflation pressure is applied to the balloon 12 (see FIG. 1A). However, some bowing 30 of the catheter 14 may nevertheless remain if the initial deflation of the balloon 12 (see FIG. 1B) resulted in the formation of permanent transverse creases 28. Permanent bowing 30 of the catheter 14 is more likely if the balloon 12 is constructed from a non-elastomeric material.

The formation of transverse creases 28 and the bowing 30 of the catheter 14 can negatively impact the use of the conventional balloon catheter 10 during certain medical procedures. For example, during angioplasty procedures, permanent creases 28 in the surface of the balloon 12 may prevent the complete or uniform compression of the tissue on the interior surface of the body canal against which the balloon 12 is expanded. This may result in a decrease in effectiveness of the angioplasty procedure.

With respect to PDT procedures, any bowing 30 of the catheter 14 can prevent accurate alignment and centering of the catheter 14 within the body lumen or canal to be treated. This is because typical PDT procedures do not allow the expanded balloon 12 to exert excess pressure or heavy contact on the interior surface of the body lumen. Thus, the balloon 12 cannot be inflated with a pressure that is sufficient to eliminate any bowing 30 of the catheter 14. The catheter 14 may consequently not be properly centered in the body lumen. As a result, effective treatment of the body lumen tissue with the therapeutic fiber optic device, which is positioned inside the catheter 14, may be inhibited.

In addition, because the distal end 16 and the proximal end 18 of the balloon 12 are both fixed to the catheter 14 at permanent (i.e., non-moveable) locations, the ability to reduce the diameter of the deflated balloon 12 may be limited, particularly if the balloon 12 is manufactured from a non-elastomeric material. In other words, the central portion 20 of the balloon 12 may not compress tightly about the catheter 14 during deflation because of the creases 28 formed in the material of the balloon 12 (see FIG. 1B). Bunching of the balloon material may likewise limit the deflated diameter or cross-section of the balloon 12. Consequently, the device may be more difficult to maneuver during ingress or egress of the device through the body's canals. In addition, the resulting “wrinkled” surface of the balloon 12 may cause irritation to body canal tissue during ingress or egress of the device and/or prevent the device from passing through the endoscope channel.

What is needed is an improved balloon catheter that overcomes the disadvantages of the conventional devices. In particular, what is needed is a balloon catheter that can be deflated to a minimal diameter for ingress and egress through the body's canals and/or an endoscope channel, that resists the formation of transverse creases in the surface of the balloon during deflation, and that resists bowing of the catheter portion located within the balloon upon inflation.

### SUMMARY OF THE INVENTION

The foregoing problems are solved and a technical advance is achieved by the balloon catheter of the present invention. The balloon catheter includes a rounded or cylindrically shaped balloon that is affixed to a catheter. The balloon includes a distal end, a proximal end and a central portion, and may be formed of a non-elastomeric material. The balloon is attached to the catheter by inserting the distal end of the catheter into and through the proximal end of the balloon until the distal end of the catheter is inserted into a portion of the distal end of the balloon. The proximal end of the balloon is then affixed to the outer wall of the catheter so as to provide an air tight seal between these components.

The distal end of the catheter is not affixed to the distal end of the balloon. In one aspect of the invention, the catheter terminates at or near the proximal end of the balloon. A stiffening member is disposed within the catheter and extends distally through the interior of the balloon and forms a slip joint connection with the distal end of the balloon. The slip joint allows the distal end of the balloon to axially move or translate with respect to the distal end of the catheter while maintaining axial alignment of the balloon relative to the stiffening member.

The above-described configuration allows the overall length of the balloon to change during inflation or deflation, the change in length of the balloon not being impeded by the predetermined length of the catheter. In addition, the above-described configuration prevents the relative axial rigidity of the catheter and stiffening member from generating any axial tensile or compressive forces in the balloon. Consequently, transverse creasing of the central portion of the balloon is eliminated or at least minimized. Moreover, the central portion of the balloon can be collapsed into a smaller diameter or cross-section for ingress or egress of the balloon catheter through the body's canals and/or the endoscope channel.

The slip joint (or the elimination of a continuous catheter connected between both ends of the balloon) also prevents balloon from generating any adverse forces in the catheter during inflation or deflation of the device. In particular, since the distal end of the balloon is not rigidly connected to the distal end of the catheter, any axial contraction or expansion of the balloon will not impart any tensile or compressive forces along the axis of the catheter, and the catheter will not be bowed or stretched as result of the inflation or deflation of the balloon. Consequently, the catheter should remain centered with respect to the cross-section of the balloon irrespective of the state of inflation of the balloon.

These and other advantages, as well as the invention itself, will become apparent in the details of construction and operation as more fully described below. Moreover, it should be appreciated that several aspects of the invention can be used with other types of balloon catheters or medical devices.

### BRIEF DESCRIPTION OF THE DRAWING

Embodiments of the present invention will now be described by way of example with reference to the accompanying drawings, in which:

FIGS. 1A-1D depict cross-sectional side views of a conventional balloon catheter in various stages of inflation and deflation;

FIG. 2 depicts a cross-sectional side view of an illustrative embodiment of a balloon catheter in accordance with the teachings of the present invention;

FIG. 3 is a cross-sectional side view of a second embodiment of a balloon catheter in accordance with the teachings of the present invention;

FIG. 4 is a cross-sectional side view of a third embodiment of a balloon catheter in accordance with the teachings of the present invention;

FIG. 5 is a cross-sectional side view of a fourth embodiment of a balloon catheter in accordance with the teachings of the present invention;

FIG. 6 is a cross-sectional side view of a fifth embodiment of a balloon catheter in accordance with the teachings of the present invention;

FIG. 7 is a cross-sectional side view of a sixth embodiment of a balloon catheter in accordance with the teachings of the present invention;

FIG. 8 is a cross-sectional side view of a seventh embodiment of a balloon catheter in accordance with the teachings of the present invention;

FIG. 9 is a cross-sectional side view of a eighth embodiment of a balloon catheter in accordance with the teachings of the present invention; and

FIG. 10 is a cross-sectional side view of an alternative configuration for the distal end portion of the balloon catheter of FIG. 8.

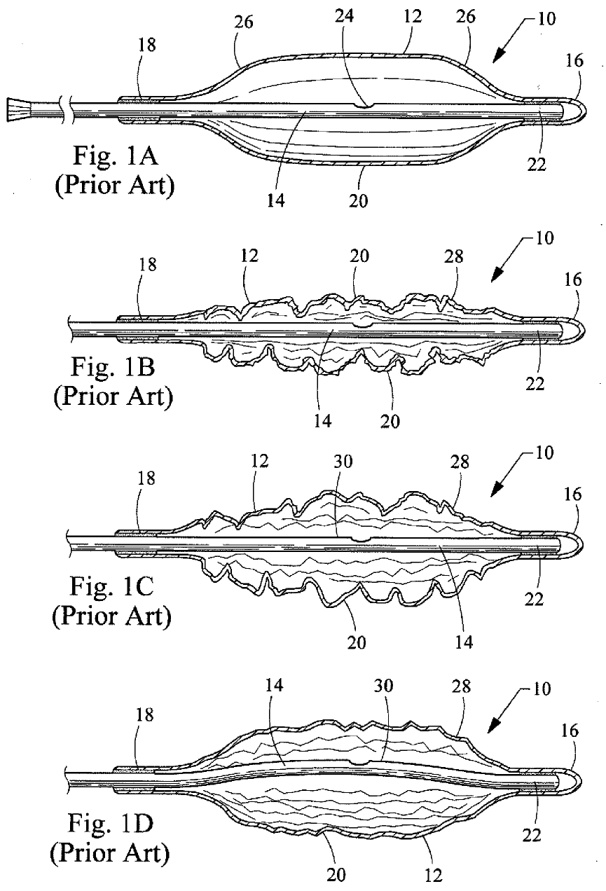

### DETAILED DESCRIPTION

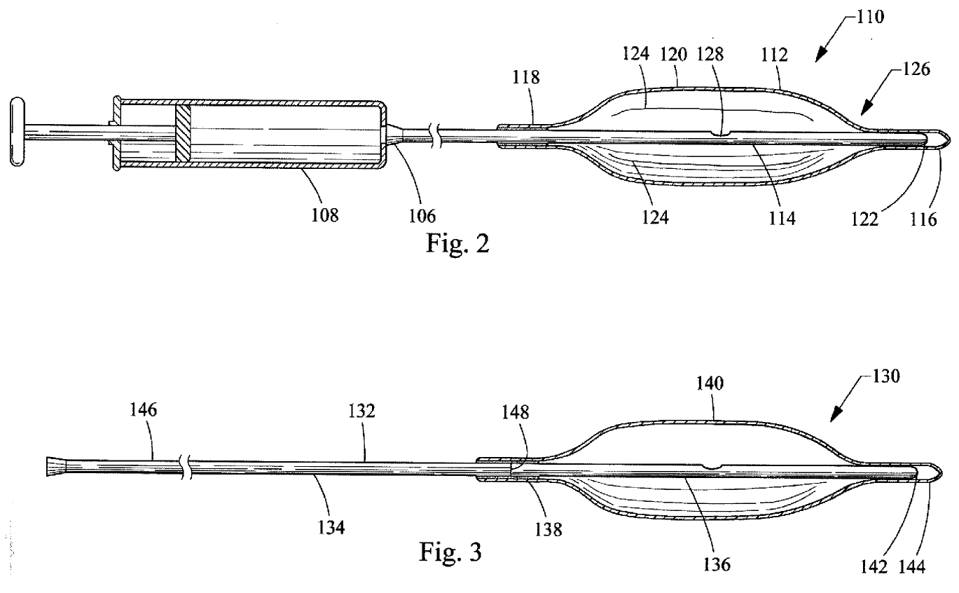

A first embodiment of a balloon catheter 110 of the present invention is depicted in FIG. 2. The balloon catheter 110 includes a rounded, oval, cylindrical, bullet or other appropriately shaped balloon 112 that is affixed to a catheter 114. The balloon 112 is typically manufactured from a non-elastomeric material (e.g., a semi-rigid or non-compliant material), and preferably comprises a translucent, transparent or optically clear film. For example, the balloon 112 could be manufactured from a biocompatible polymer such as polyamide, polyurethane, polyester, polyolefin, polyethylene terephthalate and the like.

The balloon 112, as shown in the drawings, includes a distal end 116, a proximal end 118 and a central portion 120. However, different configurations or designs can also be utilized for the balloon 112. For example, the distal end 116 and the proximal end 118 could both comprise a tubular construction so as to form a neck. The balloon 112 is attached to the catheter 114 by inserting the distal end 122 of the catheter 114 into and through the proximal end 118 of the balloon 112. The balloon 112 is then slid over the catheter 114 until the distal end 122 of the catheter 114 is inserted into a portion of the distal end 16 of the balloon 112. The proximal end 118 of the balloon 112 is then affixed to the outer wall of the catheter 114 by an adhesive, ultrasonic welding, or some other method so as to anchor and seal the proximal end of the balloon 112. In the preferred embodiment shown, the inside diameter of the proximal end 118 is sized to fit tightly or snugly over the catheter 114 so as to improve the integrity of the seal between these two components.

The distal end 122 of the catheter 114 is not affixed to the distal end 116 of the balloon 112. As shown in the drawing, the distal end 122 of the catheter 114 extends partially, but not fully, into the distal end 16 of the balloon 112 so as to form a slip joint 126 between these two components. The slip joint 126 allows the distal end 116 of the balloon 112 to axially move or translate with respect to the distal end 122 of the catheter 114. This configuration allows the overall axial or longitudinal length of balloon 112 to change during inflation or deflation without transferring tensile or compressive forces to the catheter 114. For example, when the balloon 112 is deflated, the balloon 112 tends to elongate in the axial direction as the central portion 120 is drawn inwardly towards the catheter 114, thereby moving the distal end 116 of the balloon 112 distally from or relative to the distal end 122 of the catheter 114. Since the distal end 116 of the balloon 112 is not prevented from moving axially, transverse creasing of the central portion 120 of the balloon 112 during deflation is eliminated or at least minimized. Moreover, the central portion 120 of the balloon 112 can be collapsed into a smaller diameter or cross-section for ingress or egress of the balloon catheter 110 through the body's canals and/or the endoscope channel.

The slip joint 126 also prevents the application of adverse forces on the catheter 114 by the balloon 112 during inflation or deflation of the device. In particular, since the distal end 116 of the balloon 112 is not connected to the distal end 122 of the catheter 114, any axial contraction or expansion of the balloon 112 will not impart any tensile or compressive forces onto the catheter 114. In other words, the catheter 114 will not be bowed or stretched as result of the inflation or deflation of the balloon 112. Consequently, the catheter 114 should remain centered with respect to cross-sectional area of the balloon 112 irrespective of the state of inflation of the balloon 112.

By partially extending the distal end 122 of the catheter 114 into the distal end 116 of the balloon 112, the distal end 122 of the catheter 114 can provide some lateral or transverse support to the distal end 116 of the balloon 112. This lateral support can help to guide the device, and prevent the balloon 112 from folding or collapsing, as the device is being inserted into the body's canals. The length of the distal end 116 of the balloon 112, and the position of the distal end 122 of the catheter 14 therein, should be sufficient to permit these components to freely translate with respect to each other in response to all stages of inflation and deflation of the device.

The distal end 116 of the balloon 112 is sealed so as to enclose the balloon 112. In the preferred embodiment shown, the distal end 116 of the balloon 112 is formed by inserting and sealing a small rod into the neck of the balloon 112. The distal end 116 of the balloon 112 may also be rounded to improve the ingress of the balloon catheter 110 into and through the body's canals and lumens, as well as through the channel of an endoscope. In addition, the inside diameter of the distal end 116 of the balloon 112 is slightly larger than the outside diameter of the distal end 122 of the catheter 114 so as to permit air or fluid to enter or be removed from the interior volume of the balloon 112 by passing through the distal end 122 of the catheter 114. Alternatively, an aperture 128 may be provided in the wall of the catheter 114 at a location proximal to the distal end 122, but within the interior volume of the balloon 112.

The central portion 120 of the balloon 112 may be provided with longitudinally or axially extending pleats or folds 124. These folds 124 provide creases along which the surface of the balloon 112 will fold or pleat when deflated. The folds 124 permit the central portion 120 of the balloon 112 to be collapsed to a minimal cross-sectional area or diameter, and prevent the formation of transverse or lateral creases along the same area.

The proximal end 106 of the catheter 114 is typically connected to an inflation device 108, such as a standard medical syringe. The inflation device 108 is in fluid communication with the interior of the balloon 112 via a lumen extending through the inside of the catheter 114. The catheter 114 may also comprise additional lumens through which contrast fluids or guide wires (not shown) can be passed.

A second embodiment of a balloon catheter 130 of the present invention is depicted in FIG. 3. The balloon catheter of this embodiment 130 is similar to the embodiment of the balloon catheter 110 shown in FIG. 2, but comprises a two-part catheter 132 having a relatively flexible portion 134 and a relatively rigid portion 136. The flexible portion 134 extends from approximately the proximal end 138 of the balloon 140 to the proximal end 146 of the catheter 132. The flexible portion 134 has a similar design and construction as that of the catheter 114 of the first embodiment shown in FIG. 2.

The rigid portion 136 extends from approximately the proximal end 138 of the balloon 140 to the distal end 142 of the catheter 132. In other words, the rigid portion 136 is that portion of the catheter 132 that is disposed within the balloon 140. The rigid portion 136 is less likely to sag under its own weight or the weight of the balloon 140, and may provide increased lateral support to the distal end 44 of the balloon 140. The increased rigidity of the rigid portion 136 of the catheter 132 may be particularly beneficial for use in PDT procedures, where proper centering and alignment of the therapeutic fiber optic device (not shown) within the catheter 132 is critical.

In the embodiment shown, the flexible portion 134 is connected to the rigid portion 136 at a joint 148 that is preferably located within the proximal end 138 of the balloon 140. The proximal end 138 provides reinforcement to the joint 148, as well as improving the integrity of the seal between these components.

With the exception of the two-part catheter 132 described above, the remaining components of the balloon catheter 130 of the second embodiment are the same or similar to the components of the balloon catheter 110 of the first embodiment. A detailed description of these components and their functions will consequently not be repeated here.

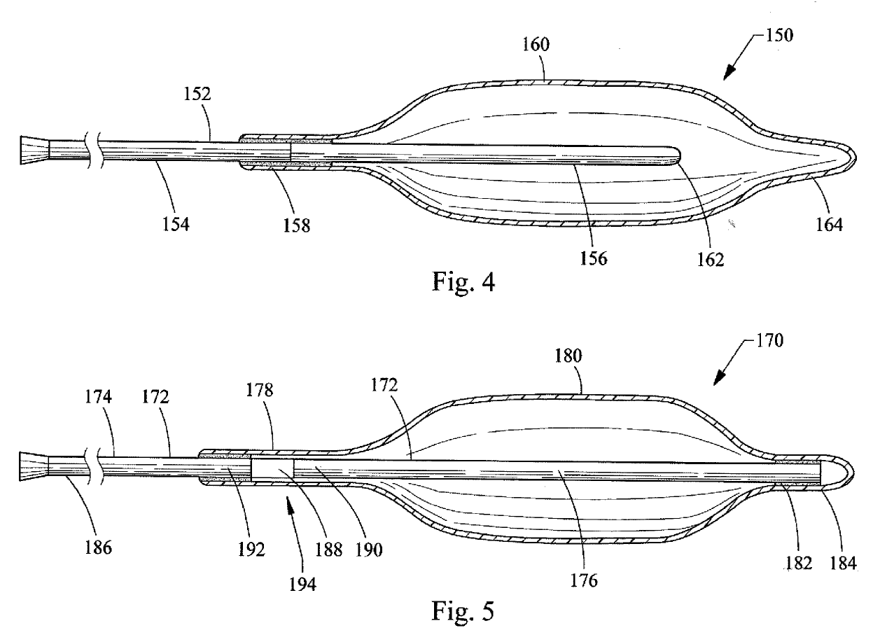

A third embodiment of a balloon catheter 150 of the present invention is depicted in FIG. 4. The balloon catheter 150 of this embodiment is similar to the embodiment of the balloon catheter 130 shown in FIG. 3 in that it also comprises a two-part catheter 152 having a flexible portion 154 and a rigid portion 156. However, the rigid portion 156 does not extend to the distal end 164 of the balloon 160. In other words, the rigid portion 156 only extends from near the proximal end 158 of the balloon 160 to part way into the interior volume of the balloon 160, and the distal end 162 of the rigid portion 156 does not form a slip joint with the distal end 164 of the balloon 160.

With the exception of the two-part catheter 152 described above, and the length of the rigid portion 156 thereof, the remaining components of the balloon catheter 150 of the third embodiment are the same or similar to the components of the balloon catheter 130 of the second embodiment. A detailed description of these components and their functions will consequently not be repeated here.

A fourth embodiment of a balloon catheter 170 of the present invention is depicted in FIG. 5. The balloon catheter of this embodiment 170 is similar to the embodiment of the balloon catheter 110 shown in FIG. 2, but comprises a segmented catheter 172 having a flexible portion 174 and a segmented or spaced apart portion 176. The flexible portion 174 extends from approximately the proximal end 178 of the balloon 180 to the proximal end 186 of the catheter 172. The flexible portion 174 has a similar design and construction as that of the catheter 114 of the first embodiment shown in FIG. 2. The distal end 192 of the flexible portion 174 is affixed to the proximal end 178 of the balloon 180 by adhesive or some other form of bonding. The segmented portion 176 can be either rigid or flexible, and either hollow or solid. In other words, the segmented portion 176 can be a rod-like length of material as opposed to a catheter-like tube since the segmented portion 176 does not necessarily need to carry fluid between the inflation device (not shown) and the balloon 180.

The distal end 182 of the segmented portion 176 is affixed to the distal end 184 of the balloon 180. The segmented portion 176 extends proximally from the distal end 182 and terminates within the proximal end 178 of the balloon 180. The proximal end 190 of the segmented portion 176 is not affixed or bonded to the proximal end 178 of the balloon 180, but is free to move axially within the proximal end 178. In other words, a slip joint 194 is formed between the proximal end 190 of the segmented portion 176 and the proximal end 178 of the balloon 180. A gap 188 is provided between the proximal end 190 of segmented portion 176 and the distal end 192 of the flexible portion 174 within the proximal end 178 of the balloon 180. This gap 188 provides room for the segmented portion 176 to move longitudinally within the proximal end 178 of the balloon 180 as the balloon 180 longitudinally contracts or elongates during inflation and deflation, as well as allowing fluid from the inflation device (not shown) to pass through the distal end 192 of the flexible portion 174 and into the interior of the balloon 180. The proximal end 178 of the balloon 180 also provides lateral support to the proximal end 190 of the segmented portion 176.

This embodiment has the advantage of allowing the balloon 180, and the segmented portion 176 of the catheter 172, to flex near the proximal end 178 of the balloon 180. This may provide increased maneuverability of the balloon catheter 170 during insertion of the device into and through the body's canals.

Of course, it should be appreciated that the segmented portion 176 could terminate short of the proximal end 178 of the balloon 180. In other words, the segmented portion 176 could extend only partially into the interior volume of the balloon 180, thereby eliminating any contact with the proximal end 178 of the balloon 180.

With the exception of the segmented catheter 172 described above, and the location of the slip joint 194 at the proximal end 178 of the balloon 180, the remaining components of the balloon catheter 170 of the fourth embodiment are the same or similar to the components of the balloon catheter 110 of the first embodiment. A detailed description of these components and their functions will consequently not be repeated here.

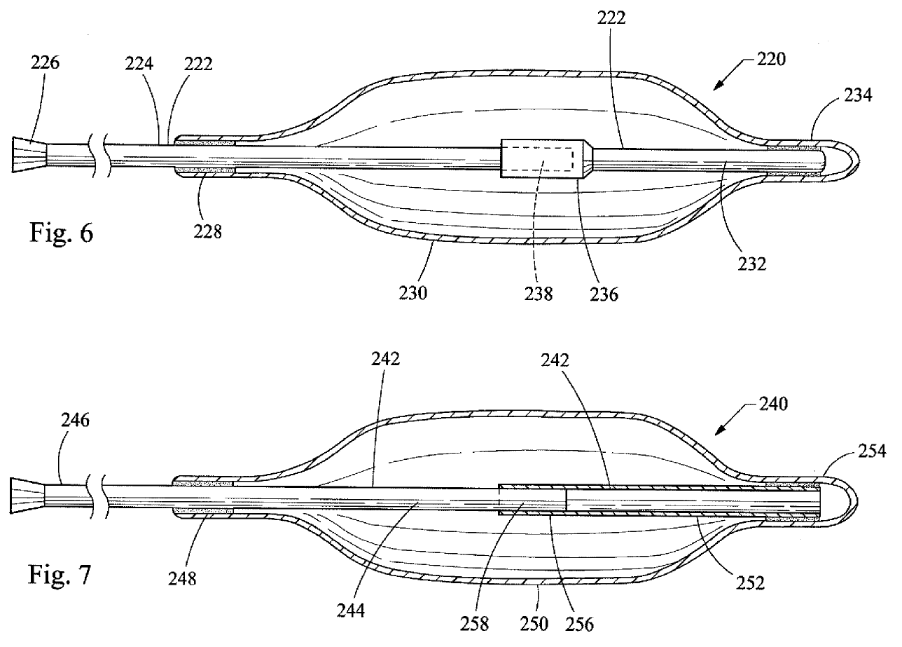

A fifth embodiment of a balloon catheter 220 of the present invention is depicted in FIG. 6. The balloon catheter of this embodiment 220 is similar to the embodiment of the balloon catheter 170 shown in FIG. 5 in that this embodiment comprises a segmented or two-piece catheter 222. However, the proximal portion 224 of the catheter 222 extends from the proximal end 226 of the catheter, through the proximal end 228 of the balloon 230, and into the interior volume of the balloon 230 where it terminates near the mid-section of the balloon 230. The proximal portion 224 of the catheter 222 is affixed to the proximal end 228 of the balloon 230.

The distal portion 232 of the catheter 222 is affixed to the distal end 234 of the balloon 230, and likewise extends into the interior volume of the balloon 230 where it terminates near the mid-section of the balloon 230. The proximal end 236 of the distal portion 232 of the catheter 222 overlaps the distal end 238 of the proximal portion 224 of the catheter 222 in a sliding arrangement. In the embodiment shown, the proximal end 236 of the distal portion 232 of the catheter 222 comprises an expanded tubular portion with an interior diameter that is slightly larger than the exterior diameter of the distal end 238 of the proximal portion 224 of the catheter 222 so as to permit relative axial movement between these two catheter components. This type of connection is often referred to as a male-female type of connection.

A sixth embodiment of a balloon catheter 240 of the present invention is depicted in FIG. 7. The balloon catheter of this embodiment 240 is similar to the embodiment of the balloon catheter 220 shown in FIG. 6 in that this embodiment comprises a segmented or two-piece catheter 242, wherein the proximal portion 244 of the catheter 242 extends from the proximal end 246 of the catheter, through the proximal end 248 of the balloon 250, and into the interior volume of the balloon 250 where it terminates near the mid-section of the balloon 250. The proximal portion 244 of the catheter 242 is affixed to the proximal end 248 of the balloon 250.

The distal portion 252 of the catheter 242 is affixed to the distal end 254 of the balloon 250, and likewise extends into the interior volume of the balloon 250 where it terminates near the mid-section of the balloon 250. The proximal end 256 of the distal portion 252 of the catheter 242 overlaps the distal end 258 of the proximal portion 244 of the catheter 242 in a sliding arrangement. In the embodiment shown, the distal portion 252 of the catheter 242 comprises a uniform tubular cross-section with an interior diameter that is slightly larger than the exterior diameter of the distal end 258 of the proximal portion 244 of the catheter 242 so as to permit relative axial movement between these two catheter components.

In the fifth and sixth embodiments (FIGS. 6 and 7), the overlapping portions of the separate catheter segments provide transverse or lateral stability to the balloon without impeding the axial expansion or contraction of the balloon. This is because the balloon is only fixedly connected to a either one of the catheter portions at single location.

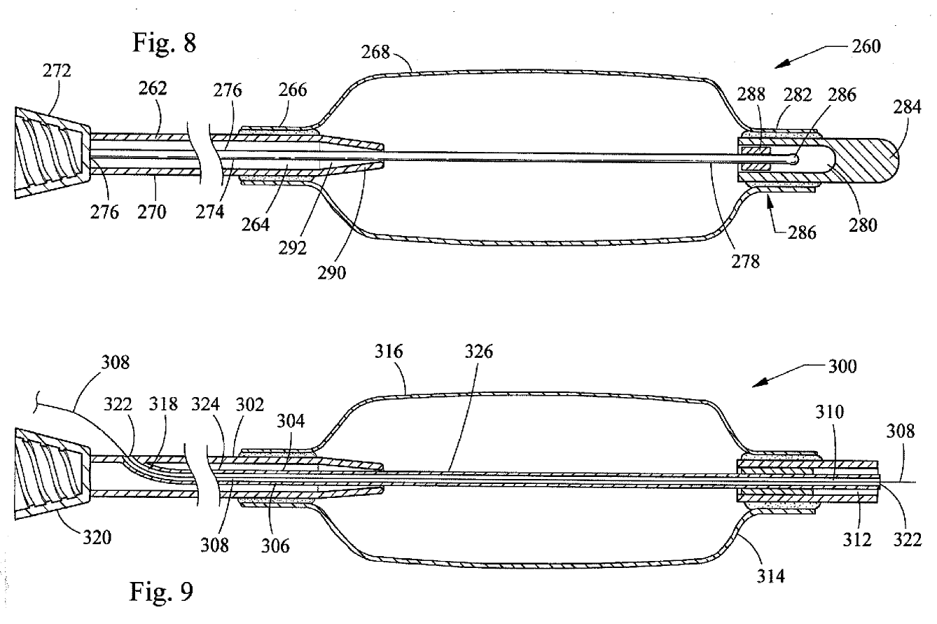

A seventh embodiment of a balloon catheter 260 of the present invention is depicted in FIG. 8. The balloon catheter 260 of this embodiment comprises a flexible elongate outer catheter 262 that is fixedly connected at its distal end 264 to the proximal end 266 of the balloon 268. The proximal end 270 of outer catheter 262 includes a luer fitting 272 that is configured to attach to an inflation device such a standard medical syringe (as shown in FIG. 2). The outer catheter 262 has a construction similar to that described in connection with the above embodiments.

The balloon catheter 260 further comprises an elongate stiffening member 274 disposed within the lumen 276 of the outer catheter 262. The diameter or cross-sectional area of the stiffening member 274 is generally less than the diameter or cross-sectional area of the lumen 276 so as to allow the passage of fluid between the luer fitting 272 (i.e., the inflation device) and the interior of the balloon 268. In other words, the diameter of the stiffening member 274 is less than that of the lumen 276 so as to create a cavity between the outside surface of the stiffening member 274 and the inside surface of the lumen 276 sufficient for the passage of an inflation lumen. Alternatively, the outer catheter 262 may comprise a separate lumen for the passage of an inflation fluid.

As illustrated in FIG. 8, the stiffening member 274 is connected at or near its proximal end 276 to the luer fitting 272. The distal end 278 of the stiffening member 274 extends distally from the distal end 264 of the outer catheter 262, through the interior of the balloon 268, and into a sleeve 280 formed in the distal end 282 of the balloon 268. In the embodiment shown, the sleeve 280 is formed by an end cap 284 fixed to the distal end 282 of the balloon 268. The end cap 284 provides an air tight seal with the balloon 268 and is rounded at its distal end to facilitate ingress of the balloon catheter 260 into and through the patient's bodily lumen and prevent the end cap 284 from puncturing or injuring the walls of the bodily lumen. The end cap 284 may be manufactured from a pliable plastic material to further promote the ingress of the balloon catheter 260 and reduce irritation that may be caused thereby.

The distal end 278 of the stiffening member 274 slidably engages with sleeve 280 to form a slip joint 286 that is similar to the slip joint 126 of the balloon catheter 110 shown in FIG. 2. As described in detail above, the slip joint 286 allows the distal end 282 of the balloon 268 to axially move or translate with respect to the distal end 278 of stiffening member 274. This configuration allows the overall axial or longitudinal length of balloon 268 to change during inflation or deflation without transferring tensile or compressive forces to either outer catheter 262 or stiffening member 274.

In the embodiment illustrated in FIG. 8, a collar or cannula 288 is disposed inside the sleeve 280. The cannula 288 has an inside diameter that is slightly greater than the outside diameter of stiffening member 274 so as to allow the stiffening member 274 to move axially or slide with respect to the cannula 288. In other words, slip joint 286 is formed by the interaction of stiffening member 274 with cannula 288. The cannula 288 aligns stiffening member 274 with the central axis of the distal end 282 of the balloon 268. A press-fit connection is utilized to dispose cannula 288 within the sleeve 280 of end cap 284. The press-fit connection is formed by manufacturing the cannula 288 to have an outside diameter that is slightly larger than the inside diameter of sleeve 280. The cannula 288 may be comprised of metal or other radiopaque material so as to provide a radiopaque reference point for accurately positioning the distal end 282 of the balloon 268 within the patient.

The distal end 278 of stiffening member 274 comprises a bead 286. The bead 286 has a rounded tip to reduce friction between the distal end 278 of stiffening member 274 and the inside surface of the sleeve 280 of end cap 284, particularly if end cap 284 has been curved by the process of inserting balloon catheter 260 into the patient's bodily lumen. The bead 286 also comprises a cross-sectional diameter that is larger than the inside diameter of cannula 288. This arrangement prevents the distal end 282 of the balloon 268 from disconnecting from the stiffening member 274 in the event that balloon 268 should rupture within the patient. More specifically, if the distal end 282 of the balloon 268 becomes separated from the remainder of the balloon 268, the bead 286 will prevent the cannula 288 from sliding off the distal end 278 of the stiffening member 274.

In the embodiment illustrated in FIG. 8, stiffening member 274 is fixedly engaged with the distal end 264 of the outer catheter 262. More specifically, the distal end 264 of the outer catheter 262 comprises a tapered guide member 290 that reduces the interior diameter of the lumen 276 of outer catheter 262 down to the outer diameter of the stiffening member 274. Alternatively, guide member 290 may be configured to permit a sliding engagement between stiffening member 274 and the distal end 264 of the outer catheter 262. The reduced diameter of the distal end of the guide member 290 aligns the stiffening member 274 with the central axis of the proximal end 264 of the balloon 268. The guide member 290 comprises one or more openings or ports 292 to allow the passage of inflation fluid between the lumen 276 of the outer catheter 262 and the interior of the balloon 268. The guide member 290 may be comprised of metal or other radiopaque material so as to provide a radiopaque reference point for accurately positioning the proximal end 266 of the balloon 268 within the patient.

Stiffening member 274 comprises a solid wire that may have a stiffness or resistance to bending that is greater than the stiffness or resistance to bending of the outer catheter 262. In addition to maintaining alignment of the distal end 286 of the balloon 268, the stiffening member 274 enhances the overall stiffness and pushability of balloon catheter 260. In other words, overall stiffness and pushability of balloon catheter 260 is achieved by the combination of the stiffening member 274 and the outer catheter 262. The stiffening member 274 may also provide a radiopaque reference line for accurately positioning the central axis (or centerline) of the balloon 268 within the patient.

The stiffening member 274 may have either a circular or non-circular cross-section. In particular, a non-circular cross-section (e.g., triangular or star-shaped) may be utilized to increase the strength or stiffness of the stiffening member 274 without inhibiting the flow of inflation fluid through the lumen 276 of the outer catheter 262. The stiffening member 274 may also comprise hollow cross-section with a lumen disposed therein. As will be explained below in connection with the eighth embodiment shown in FIG. 9, a lumen extending through the stiffening member 274 could be used to accommodate a wire guide.

The stiffening member 274 may have non-uniform properties along the length thereof. For example, the stiffening member 274 may be tapered (e.g., having a decreasing cross-section) so as to have stiffness that decreases from its proximal end 276 to its distal end 278. The stiffening member 274 may also be manufactured from different materials having different physical properties. A stiffening member 274 having a decreasing stiffness along the length thereof would provide the balloon catheter 260 with greater stiffness near the proximal end 270 where the ability to push the balloon catheter 260 (i.e., “pushability”) is most important, while providing greater flexibility near the distal end 264 where the ability to guide the balloon catheter 260 around tortuous pathways is most important. The stiffening member 274 may also be manufactured from different materials having different physical properties.

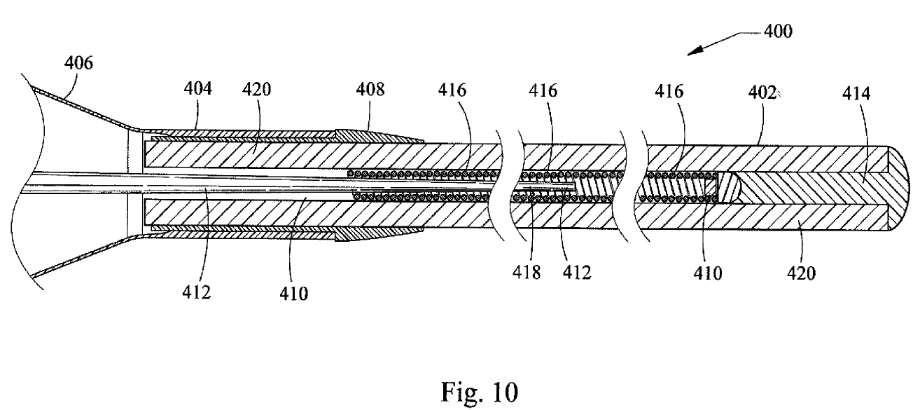

FIG. 10 illustrates an alternative configuration for the distal end portion of the balloon catheter 260 of FIG. 8. In this particular embodiment, the balloon catheter 400 comprises an elongated end cap 402 that is affixed to the distal end 404 of the balloon 406 by an adhesive 408. The elongated end cap 402 is longer than the end cap 284 of the embodiment shown in FIG. 8, and is configured to reduce trauma to the patient during insertion and advancement of the balloon catheter 400 into and through the patient. In the particular embodiment illustrated, the elongated end cap 402 comprises a polyurethane tube 420 having a length of approximately 3 cm, an outside diameter of approximately 0.080 inches+/−0.001 inches, and an inside diameter of approximately 0.031 inches+/−0.001 inches. The inside diameter of the polyurethane tube 420 is generally open and forms a sleeve 410 into which the distal end of the stiffening member 412 is slidably disposed. The polyurethane tube 420 preferably comprises a pellethane material. It has been determined that a polyurethane (e.g., pellethane) tube 420 having the above-described dimensions provides an end cap 402 having a desirable amount of flexibility so as to form an atraumatic end cap 402. However, other materials and dimensions may be utilized to achieve the desired flexibility.

An adhesive plug 414 fills the distal most portion of the sleeve 410 so as to seal the interior volume of the balloon 406. As illustrated in FIG. 10, the adhesive plug 414 is rounded and covers the distal most portion of the end cap 402 (polyurethane tube 420) so as to form an atraumatic tip. The shape of the adhesive plug 414 may be formed by first filling the distal most portion of the sleeve 410 and covering the distal end of the polyurethane tube 420 with adhesive, then shaping the adhesive so as to form the desired shape, which is preferably rounded. Shaping of the adhesive may be accomplished by heating and then manipulating the adhesive, or by removing excess adhesive (e.g., by cutting or machining), until the desired shape is obtained. Alternatively, a plug may be separately formed and inserted into the distal end of the polyurethane tube 420.

A transition member, such as coil spring 416 is embedded within a central portion of the sleeve 410 of the end cap 402. The coil spring 416 controls bending and flexibility and of the end cap 402, and prevents the end cap 402 from kinking as the balloon catheter 400 is introduced into the patient. In other words, the coil spring 402 provides the end cap 402 (i.e., the polyurethane tube 420) with the desired rigidity and flexibility to insure that the end cap 402 will flex along a gradual arc as the balloon catheter 400 is advanced through the bodily lumens of the patient. The coil spring 416 also prevents the distal end of the stiffening member 412 from puncturing or otherwise engaging the inside surface of the end cap 402, particularly when the end cap 402 is in a flexed or curved configuration. As will be explained below, the coil spring 416 also provides a transition in stiffness between the relatively stiff stiffening member 412 and the relatively flexible end cap 402. This transition in stiffness prevents or at least inhibits kinking of the end cap 402. Kinking of the end cap 402 can cause the sleeve 410 to collapse, thereby inhibiting movement of the stiffening member 412 relative to the end cap 402. The coil spring 416 also reduces frictional forces between the distal end of the stiffening member 412 and the inside surface of the sleeve 410.

As shown in FIG. 10, the distal end of the stiffening member 412 terminates near a mid-point of the coil spring 416 when the balloon 406 is in an inflated configuration. The coil spring 416 has a length sufficient to maintain the distal end of the stiffening member 412 between the ends of the coil spring 416 irrespective of the deflated, inflated, or partially inflated configuration of the balloon 406. In the particular embodiment illustrated, the coil spring 416 is comprised of 304 stainless steel flat coil, and has approximate length of 5 cm, an outside diameter of approximately 0.030 inches, and an inside diameter of approximately 0.022-0.023 inches. The above described dimensions allow the coil spring 416 to be press fit into the sleeve 410 of the polyurethane tube 420. However, other methods of embedding or affixing the coil spring 416 into the interior of the end cap 402 can be employed. In addition, other types of transition members can be utilized as an alternative to coil spring 416. For example, the transition member may comprise a metal cannula having the desired properties, such as a hypo-tube that has been spirally cut to provide the desired flexibility.

The stiffening member 412 of this embodiment comprises a nitinol wire having an overall length of approximately 242 cm+/−1 cm, and an outside diameter of approximately 0.026-0.027 inches along the proximal portion hereof. The distal most portion of the stiffening member 412 comprises a tapered portion 418 having an outside diameter of approximately 0.010 inches+/−0.001 inches at the distal end of the stiffening member 412. The tapered portion 418 is generally confined to that portion of the stiffening member 412 that is disposed within coil spring 416. However, the tapered portion 418 may extend along a larger portion of the stiffening member 412. The tapered portion 418, along with coil spring 416, provides for a gradual change (i.e., reduction) in stiffness of the end cap 402 from the proximal end of the end cap 402 to the distal end of the end cap 402. As explained above, this gradual change (or transition) in stiffness prevents or at least inhibits kinking of the end cap 402 during introduction of the balloon catheter 400 into the patient.

The enhanced features of the end cap 402 illustrated in FIG. 10 provide a balloon catheter 400 that is particularly suitable for dilating strictures in the esophagus, pylorus, and colon. However, it should be understood that balloon catheter 400 may be employed in other types of medical procedures. In addition, it should be understood that end cap 402 may be incorporated into any of the other embodiments described herein.

An eighth embodiment of a balloon catheter 300 of the present invention is depicted in FIG. 9. The balloon catheter 300 of this embodiment is similar to the embodiment of the balloon catheter 260 shown in FIG. 8 in that this embodiment comprises an outer catheter 302 and separate inner member 304 disposed therein. However, in the balloon catheter 300 of this embodiment, inner member 304 comprises a cannula or catheter having an inner lumen 306 adapted to receive a wire guide 308. The distal end 310 of inner member 304 is bonded to end cap 312 at the distal end 314 of the balloon 316. The proximal end 318 of inner member 304 passes through or is attached to the wall of outer catheter 302 near luer fitting 320. Ports 322 are provided at each end 310, 318 of inner member 304 to provide access for the wire guide 308 into and out of the inner lumen 306. Other features of this embodiment are similar to the other embodiments described above and need not be repeated here.

In this embodiment, outer catheter 302 and inner member 304 are each fixedly connected to balloon 316 and to each other. As a consequence, outer catheter 302 and/or inner member 304 are configured to accommodate any lengthening or shortening of the balloon 316 caused by the inflation or deflation thereof. In other words, balloon catheter 300 is configured so that the length of outer catheter 302 and/or inner member 304 will respond to and accommodate any changes in the length of the balloon 316. Because the overall length of outer catheter 302 and inner member 304 is much greater than the length of balloon 316, the total axial expansion or contraction that must be accommodated by the outer catheter 302 and/or inner member 304 is spread out over a relatively long distance as incrementally much smaller than that of the balloon 316.

In the embodiment illustrated in FIG. 9, inner member 304 comprises a proximal section 324 and a distal section 326 having different physical or material properties. The proximal section 324 is constructed of a flexible material or otherwise configured to readily expand or contract in length (relative to outer member 302) in response to changes in the length of the balloon 316. In contrast to the proximal section 324, the distal section 326 is constructed of a relatively rigid material so as to provide lateral support to the distal end 314 of the balloon 316. The more rigid distal section 326 may also extend proximally of the balloon 316 to provide additional stiffness to the outer catheter 302. The portion of the outer catheter 302 adjacent to the more flexible proximal section 324 of the inner member 304 may be stiffened to avoid weak areas that may be prone kinking. Stiffening of the outer catheter 302 can be accomplished by, for example, increasing the cross-sectional area of the outer catheter 302 or including a separate stiffening member (not shown).

Alternatively, outer catheter 302 may comprise a material of increased elasticity that will readily elongate in response changes in the length of the balloon 316. For example, the outer catheter 302 (or a portion thereof) may be constructed of a flexible material or otherwise configured to readily expand or contract in length (relative to inner member 304) in response changes in the length of the balloon 316. In such an embodiment, the inner member 304 may be constructed of a relatively rigid material so as to provide stiffness or lateral support to the outer catheter 302 as well as to the distal end 314 of the balloon 316. In other words, the inner member 304 will act as the primary stiffening member for balloon catheter 300.

The balloon catheter 300 of this embodiment is adapted for use in medical procedures wherein a wire guide 308 is pre-positioned in the patient's bodily lumen. In such a procedure, the proximal end of wire guide 308, which extends outside of the patient, is inserted through port 322 and into inner lumen 308 of distal end of balloon catheter 300 (i.e., into the distal end 312 of inner member 306). The balloon catheter 300 is then pushed over the wire guide 308 until the balloon 316 is positioned at the desire location within the patient. The wire guide 308, which may have been previously positioned within the patient during an earlier part of the medical procedure, allows the balloon catheter 300 to be quickly inserted and guided into the patient. The balloon 316 is then inflated as described above in connection with the other embodiments.

It should be appreciated that the wire guide 308 must be long enough so that the portion of the wire guide 308 extending out of the patient is longer than the overall length of the inner member 304 of the balloon catheter 300. This length is necessary so that the proximal end of wire guide 308 will extend out of inner lumen 308 at the proximal end 318 (and proximal port 322) of inner member 304 prior to the distal end 310 of inner member 304 (or end cap 312) is inserted into the patient. This allows the wire guide 308 to be grasped and held in position at all times while the balloon catheter 300 is being fed onto the wire guide 308 and inserted into the patient.

In the embodiment illustrated in FIG. 9, the proximal end 318 (and proximal port 322) of inner member 304 is located relatively near connector 320. Inner member 304 therefore extends along a substantial portion of balloon catheter 300. Catheter devices having a wire guide lumen extending along substantially the entire overall length of the device are commonly referred to as over-the-wire devices. However, a shorter inner member 304 could be utilized. For example, the proximal end 318 (and proximal port 322) of inner member 304 could be located much closer to the balloon 316. Alternatively, additional ports 322 could be provided along the outer catheter to give access to the inner lumen 308 at intermediate locations. In such embodiments, the wire guide 308 would exit inner lumen 308 at a location near the proximal end of the balloon 316. The arrangement requires a much shorter portion of the wire guide 308 to extend out of the patient since the length of the inner lumen 308 is much shorter than the length of the balloon catheter 300. Catheter devices having a shorter wire guide lumen, or having intermediate access to the wire guide lumen, are commonly referred to as rapid exchange devices. Although the proximal port 322 is shown extending through the side wall of the outer catheter 302 and slightly distal of luer fitting 320, it should be appreciated that the location of port 322 and luer fitting 320 could be reversed.

Any other undisclosed or incidental details of the construction or composition of the various elements of the disclosed embodiments of the present invention are not considered to be critical to the achievement of the advantages of the present invention, so long as the elements possess the attributes required to perform as disclosed herein. The selection of these and other details of construction are believed to be well within the ability of one of ordinary skill in the relevant art in view of the present disclosure. Illustrative embodiments of the present invention have been described in considerable detail for the purpose of disclosing practical, operative structures whereby the invention may be practiced advantageously. The designs described herein are intended to be exemplary only. The novel characteristics of the invention may be incorporated in other structural forms without departing from the spirit and scope of the invention.

What is claimed is:

1. A balloon catheter comprising:
an inflatable balloon comprising a balloon wall defining an interior volume, the balloon further comprising a distal end, a proximal end, and a central portion disposed therebetween;
a catheter comprising an elongated shaft extending along an axis between a distal end portion and a proximal end portion, the proximal end portion comprising a connector configured to engage an inflation device, the distal end portion fixedly connected to the proximal end of the balloon, and a lumen extending though the shaft and in fluid communication with the interior volume of the balloon;
an end cap fixedly connected to the distal end of the balloon, the end cap comprising a sleeve extending partially therethrough, sleeve being defined by an interior volume of the end cap; and
a stiffening member extending distally from the distal end portion of the catheter and through the interior volume of the balloon, the stiffening member being slidably engaged with the sleeve of the end cap,
wherein a transition member is fixedly disposed within the sleeve of the end cap, the transition member comprising a lumen that is slidably engaged with a distal end of the stiffening member,
wherein movement of the distal end of the balloon relative to the proximal end of the balloon is not restrained by the catheter,
wherein axial movement of the distal end of the balloon relative to the proximal end of the balloon in a direction generally parallel to the axis of the shaft is not restrained by the stiffening member, and
wherein transverse movement of the distal end of the balloon relative to the proximal end of the balloon in a direction generally perpendicular to the axis of the shaft is restrained by the stiffening member.

2. The balloon catheter according to claim 1 wherein the balloon has a deflated axial length when deflated, and an inflated axial length when inflated, the deflated axial length and the inflated axial length each being defined by the distance between the proximal end and the distal end of the balloon, the deflated axial length being different than the inflated axial length.

3. The balloon catheter according to claim 1 wherein the balloon has a deflated axial length when deflated, and a partially inflated axial length when partially inflated, the deflated axial length and the partially inflated axial length each being defined by the distance between the proximal end and the distal end of the balloon, the deflated axial length being different than the partially inflated axial length.

4. The balloon catheter according to claim 1 wherein the balloon has a partially inflated axial length when partially inflated, and a fully inflated axial length when fully inflated, the partially inflated axial length and the fully inflated axial length each being defined by the distance between the proximal end and the distal end of the balloon, the partially inflated axial length being different than the fully inflated axial length.

5. The balloon catheter according to claim 1 wherein the balloon wall comprises one of a non-elastic material, a non-compliant material, and a semi-rigid material.

6. The balloon catheter according to claim 1 wherein the balloon wall comprises axially oriented creases or pleats to facilitate radial compression of the balloon when deflated.

7. The balloon catheter according to claim 1 wherein the stiffening member comprises a proximal portion extending along and generally parallel to the shaft of the catheter.

8. The balloon catheter according to claim 7 wherein the proximal portion of the stiffening member is disposed within the lumen of the shaft of the catheter, and wherein a proximal end of the proximal portion of the stiffening member is fixedly connected to the proximal end portion of the catheter.

9. The balloon catheter according to claim 1 wherein the end cap comprises a polyurethane tube fixedly connected to the distal end of the balloon, and the transition member is embedded within a lumen of the polyurethane tube.

10. The balloon catheter according to claim 9 wherein the transition member has an axial length that is less than an axial length of the polyurethane tube.

11. The balloon catheter according to claim 9 wherein a distal end portion of the lumen of the polyurethane tube is sealed.

12. The balloon catheter according to claim 11 wherein the distal end portion of the lumen of the polyurethane tube is sealed with an adhesive.

13. The balloon catheter according to claim 9 wherein the transition member is configured to prevent the distal end of the stiffening member from engaging an interior surface of the polyurethane tube.

14. The balloon catheter according to claim 1 wherein the distal end of the stiffening member comprises a tapered portion.

15. The balloon catheter according to claim 14 wherein the tapered portion of the stiffening member is slidably disposed within the transition member.

16. The balloon catheter according to claim 1 wherein the stiffening member comprises nitinol.

17. The balloon catheter according to claim 1 wherein the sleeve comprises a distal terminus that is spaced away from the distal end of the stiffening member so as to permit axial movement of the distal end of the stiffening member relative to the distal terminus of the sleeve.

18. The balloon catheter according to claim 1 further comprising an inflation device for inflating or deflating said balloon, said inflation device being attached to the connector on the proximal end portion of the catheter.

19. The balloon catheter according to claim 18 wherein the connector comprises a female luer fitting, and further wherein the inflation device comprises a syringe having a male luer fitting, the male luer fitting being engaged with the female luer fitting.

20. The balloon catheter according to claim 1 wherein the transition member comprises a coil spring that is embedded within the sleeve of the end cap.
